(19) United States
(12) Patent Application Publication
(10) Pub. No.: US 2011/0130655 A1
(43) Pub. Date: Jun. 2, 2011

(54) IMPLANTABLE DEVICES AND METHODS FOR EXTERNAL BEAM RADIATION TREATMENTS

(76) Inventors: Eric D. Nielson, Bellevue, WA (US); Steven C. Dimmer, Bellevue, WA (US); David N. Krag, Shelburne, VT (US); Timothy P. Mate, Bellevue, WA (US); Roland Perez, Seattle, WA (US); Fergus Quigley, Seattle, WA (US)

(21) Appl. No.: 12/956,820

(22) Filed: Nov. 30, 2010

Related U.S. Application Data
(63) Continuation of application No. 12/669,950.
(60) Provisional application No. 60/951,172, filed on Jul. 20, 2007.

(51) Int. Cl. A61B 5/05

(52) U.S. Cl. 600/426

(57) ABSTRACT

Implantable devices and methods for external beam radiation treatments are disclosed. One embodiment of an implantable device for guided radiation therapy comprises an active marker configured to be positioned within the patient and to transmit a non-ionizing wireless signal in response to a non-ionizing wirelessly transmitted source energy. The device further includes a fastening unit coupled to the active marker and configured to (a) hold the marker within a desired distance of a target in the tissue and (b) inhibit deformation of tissue from moving the active marker relative to the target.

### TECHNICAL FIELD

The present invention is related to radiation oncology and, more specifically, to accurately determining the location of a target for delivering external radiation.

### BACKGROUND

Cancer begins in the cells of a patient and forms malignant tumors that are often treated by surgical resection. Such surgical treatments attempt to remove as much of a tumor as possible, but cancerous cells infiltrate into the tissue adjacent the tumor such that there is no clear boundary. Also, certain procedures seek to limit the treatment margin around the tumor to reduce the amount of healthy tissue removed from the patient. In breast cancer, for example, patients prefer to limit the size of the lumpectomy resection to avoid excessive reduction or non-uniformities of the breast. Both of these factors limit the efficacy of surgical procedures for treating cancer. As such, radiation therapy has become a significant and highly successful process for treating breast cancer, lung cancer, brain cancer, and many other types of localized cancers. Radiation therapy is particularly useful for treating (a) tissue after resecting a tumor, (b) centrally located tumors, and/or (c) small cell tumors that cannot be surgically resected. Radiation therapy can also be used as a palliative treatment when a cure is not possible.

Breast cancer has recently been treated by surgically resecting cancerous breast tissue and subsequently treating the remaining tissue surrounding the resection cavity using radiation. Proxima Corporation and Xoft, Inc. have developed breast brachytherapy devices and systems for selectively irradiating the portion of the tissue surrounding the resection cavity created by a lumpectomy. The existing breast brachytherapy devices have a balloon configured to be implanted in the cavity within the breast and an internal radiation source that can be placed within the balloon. After performing a lumpectomy, the balloon is inserted into the surgical cavity and inflated until the balloon presses against the tissue. The balloon is typically left in the patient for approximately five days over which two radiation treatments per day are performed. Each radiation treatment includes inserting the radiation source into the balloon and activating the radiation source to deliver ionizing radiation for approximately 10-15 minutes. After all of the radiation treatments have been performed during the multi-day course of treatment, the balloon is deflated and removed from the patient.

Breast brachytherapy procedures, however, can be challenging. For example, it may be difficult to determine whether the balloon has been inflated accurately and to monitor the balloon to ensure that the balloon has maintained the desired size and adequate conformance within the resection cavity throughout the multi-day course of treatment. The size of the balloon is currently determined by instilling radiopaque contrast into the balloon and measuring a resulting CT or X-ray image using a ruler. The patient must accordingly undergo a CT scan or another type of X-ray to obtain the image, and then a practitioner must evaluate the image to determine whether the balloon is at the desired size. This is time-consuming and expensive, and it should be performed each day during the course of treatment. This process also exposes the patient to additional radiation.

Breast brachytherapy may also have disadvantages associated with using an internal radiation source. For example, the balloon may move within the lumpectomy cavity over the course of treatment, which can cause the internal radiation source to over-irradiate some areas and under-irradiate other areas. Many existing systems do not detect the relative position between the balloon and the breast to mitigate this problem. Moreover, when the radiation source is asymmetrically positioned within the balloon (e.g., spaced apart from a rotational center line of the balloon), the rotational orientation of the balloon within the lumpectomy cavity can cause the radiation source to be located at an undesirable position relative to the tissue. Conventional techniques also do not identify the rotational orientation of the balloon. This can be problematic because after the balloon has been implanted, it can move after over the course of treatment, or the balloon may not inflate as planned. Conventional breast brachytherapy systems are also relatively large because they must contain both a balloon and an internal radiation source. Many patients are not comfortable with having a radiation source within their body or with having a large catheter projecting from their body for a number of days, and, therefore, a sizable number of patients elect not to undergo breast brachytherapy. Moreover, the balloon requires a relatively large member to extend externally out of the patient, which can increase the risk of infection. Additional challenges for a balloon-based therapy include the requirement for multiple balloon sizes and shapes to fit the possible range of resection cavities and the possible dose inhomogeneity.

In light of the challenges associated with breast brachytherapy procedures, partial breast irradiation using an external radiation beam has been proposed. Although radiation beams, such as Three-Dimensional Conformal Radiation Therapy beams, can be shaped to conform to the target tissue, it is still difficult to use external beam radiation to treat the tissue around the resection cavities in many applications. For example, the size and shape of the cavity may change over the typical multi-day period for external beam radiation treatments, or the treatment target may move during the treatment sessions. Several potential treatments have proposed using balloons or scaffolds to stabilize the tissue, but balloons may still suffer from the challenges explained above. As such, there is a need for improving external beam radiation for partial breast irradiation and other procedures that seek to irradiate controlled treatment margins around resection cavities.

### BRIEF DESCRIPTION OF THE DRAWINGS

FIG. 1 is a side view illustrating an implementation of a system for performing partial breast irradiation.

FIG. 2 is a schematic view of an implantable device for use with a system for guided radiation therapy.

FIG. 3A is a side view of a specific embodiment of an implantable device for guided radiation therapy, and FIG. 3B is a cross-sectional view of a portion of the implantable device of FIG. 3A. FIG. 3C and FIG. 3D are specific alternative embodiments of an implantable device for guided radiation therapy.

FIG. 4 is a cross-sectional view of an embodiment of an implantable device for guided radiation therapy loaded in an introducer.

FIG. 5 is a schematic view of an embodiment of operating a system for guided radiation therapy.

FIG. 6 is a graph illustrating an example of a force curve for explanting the implantable device.

FIG. 7 is a schematic side view of another embodiment of an implantable device for guided radiation therapy.

FIGS. 8A-8G are side views of additional embodiments of decoupling elements used in implantable devices for guided radiation therapy.

FIGS. 9A and 9B are side views of additional embodiments of markers for implantable devices used in guided radiation therapy.

FIGS. 10A and 10E are side views of additional embodiments of stability elements used in implantable devices for guided radiation therapy.

### DETAILED DESCRIPTION

### Overview

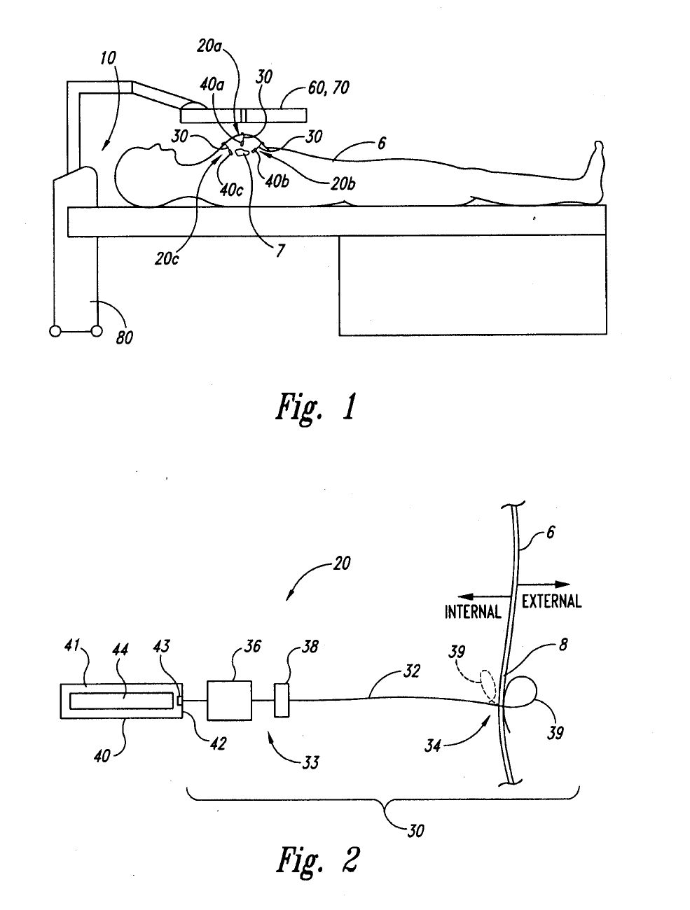

Specific details of several embodiments of the disclosure are described below with reference to implantable devices and methods for external beam radiation treatments. Although many of the embodiments are described below with respect to partial breast irradiation systems and procedures for treating resection cavities, the systems and implantable devices can be used in other procedures for treating other indications. Moreover, several other embodiments of the invention can have different configurations, components, or procedures than those described in this section. For instance, the apparatus and methods can have one or more markers or other components for use in external beam radiation therapy procedures as described in U.S. patent application Ser. Nos. 11/165,843, filed on 24 Jun. 2005, and 11/166,801, filed on 24 Jun. 2005, both of which are incorporated herein by reference. In other instances, well-known structures associated with target locating and tracking systems have not been shown or described in detail to avoid unnecessarily obscuring descriptions of the embodiments of the invention. A person of ordinary skill in the art, therefore, will accordingly understand that the invention may have other embodiments with additional elements, or the invention may have other embodiments without several of the features shown and described below with reference to FIGS. 1-10E.

One embodiment of an implantable device for guided radiation therapy comprises an active marker having a circuit configured to be implanted in a patient and transmit a wirelessly transmitted location signal from within the patient in response to a wirelessly transmitted source energy. In an alternative embodiment, the active marker transmits a location signal along a wire. The implantable device can further include a fastening unit having a first portion coupled to the active marker and a second portion configured to be at least proximate to the dermis of the patient. The marker, for example, can comprise a leadless marker having a circuit with a core and a coil around the core.

Another embodiment of an implantable device for guided radiation therapy comprises an active marker configured to be implanted in tissue of the patient and to transmit a non-ionizing wireless signal in response to a non-ionizing wirelessly transmitted source energy. The implantable device can further include a stability element coupled to the marker and an explant line. The stability element is configured to hold the marker at an implant location (e.g., known location) with respect to a target in the tissue. The explant line has a first portion coupled to the marker and/or the stability element and a second portion configured to be at least proximate to the dermis of the patient. The implantable device can, optionally, also include a decoupling element coupled to the explant line and spaced proximally apart from the stability element. The decoupling element, for example, can be configured to inhibit deformation of tissue from moving the active marker relative to the target.

Another embodiment of an implantable device for guided radiation therapy comprises an active marker configured to be positioned within the patient and to transmit a non-ionizing wireless signal in response to a non-ionizing wirelessly transmitted source energy. The device further includes a fastening unit coupled to the active marker and configured to (a) hold the marker within a desired distance of a target in the tissue and (b) inhibit deformation of tissue from moving the active marker relative to the target.

### 1. Embodiments of Localization Systems and Implantable Devices

FIG. 1 is a side view of a localization system 10 with an implantable device 20 for facilitating radiation treatment of a target in accordance with an embodiment of the invention. In the embodiment shown in FIG. 1, the system 10 has three individual implantable devices 20 (identified separately by reference numbers 20a-c) implanted in a patient 6 relative to a resection cavity 7. Each implantable device 20 can include a fastening unit 30 and a marker 40 coupled to the fastening unit 30. In the embodiment shown in FIG. 1, the implantable devices 20a-c include markers 40a-c, respectively. The markers 40 can be active markers configured to transmit independent location signals in response to an energy source located external to the body of the patient (e.g., outside the dermis of the patient). For example, the markers 40 can be wireless active sensors that wirelessly transmit location signals in response to wirelessly transmitted excitation signals. Such wireless active markers can comprise a magnetic transponder as described in U.S. patent application Ser. Nos. 11/243,478 and 11/166,801, both of which are incorporated herein by reference in their entirety. The localization system 10 can further include an excitation source 60 that wirelessly transmits excitation signals to the markers 40, a sensor assembly 70 that measures the location signals wirelessly transmitted from the markers 40, and a controller 80.

In several embodiments, one or more implantable devices 20 are implanted in the patient 6 such that the markers 40 are at least proximate to the resection cavity 7. The markers 40 are accordingly associated with the resection cavity such that the markers 40 move based on the position, rotation, and/or expansion-contraction of the resection cavity 7. In the embodiment shown in FIG. 1, the three markers 40a-c are associated with the resection cavity, but a single marker, two markers, or more than three markers can be used depending on the particular application. Two markers, for example, may be desirable because the target can be located accurately and the relative displacement between the markers over time can be used to monitor the status and position of the resection cavity 7.

The localization system 10 determines the actual location of the markers 40 in a three-dimensional reference frame when the markers are within or on the patient 6. In a particular embodiment of the system 10 illustrated in FIG. 1, the localization system 10 tracks the three-dimensional coordinates of the markers 40a-c in real time to an absolute external reference frame during the setup process and while irradiating the patient to mitigate collateral effects on adjacent tissue outside the treatment margin and to ensure that the desired dosage is applied to the target tissue.

Several embodiments of the implantable device 20 enable accurate determination of the size of the resection cavity 7 within the breast of the patient without taking expensive CT images and manually assessing the images. This aspect is very useful because the shape and size of the resection cavity 7 may change over the course of the treatment. This change could cause the external beam radiation to irradiate healthy tissue but miss targeted tissue. By localizing the relative positions of the markers 40, changes in the size and shape of the resection cavity 7 can be determined before, during, and after each treatment session to ensure that the desired dose of radiation is accurately delivered to the correct tissue.

Several embodiments of the implantable device 20 can also track movement of the resection cavity or other treatment target throughout the course of therapy to accurately deliver external beam radiation within the treatment margin. Breast tissue, for example, is soft and pliable such that it may be difficult to hold the treatment target at the isocenter of the external radiation beam. The breast is also likely to move during treatment because of thoracic expansion/contraction caused by respiration. Several embodiments of the implantable device 20 are also useful for detecting movement of the patient or other displacement of the breast in real time during therapy. As a result, the implantable device 20 is expected to provide accurate measurements to confirm the status and the location of the treatment target throughout the course of therapy.

Several embodiments of the implantable device 20 also track the rotational orientation of the resection cavity or other target site relative to the body or the radiation beam throughout the course of treatment. The rotational orientation of the target site may be important in several applications because resection cavities and other targets are generally not spherical such that the rotational orientation affects the profile of the treatment margin relative to the position of the external beam. The markers 40 can be tracked or otherwise located using the localization system 10 to determine rotational orientation of the target relative to the external beam.

FIG. 2 is a schematic view of an embodiment of the implantable device 20 implanted internally of a dermis 8 of the patient 6. In this embodiment, the fastening unit 30 has a line 32 with a first portion 33 coupled to the active marker 40 at a distal area and a second portion 34 at a proximal area at least proximate to the dermis 8. The line 32 can be a flexible, elongated tether having a dielectric exterior to prevent currents from becoming induced if the line forms a closed loop and affecting the localization signals in applications that use active markers 40 which transmit alternating magnetic fields. The line 32, for example, can be a thin mono-filament, multifilament, cable, and/or combination thereof, made from a dielectric material (e.g., a polymer), a metal wire, and/or a ribbon with a dielectric coating. In still other embodiments, the line 32 can have an exterior surface configured to prevent tissue incorporation (e.g., tissue attachment). In other applications, the line 32 may be a conductive filament or wire without a dielectric coating. The fastening unit 30 can also have a stability element 36 at the first portion 33 of the line 32, a decoupling element 38 at the line 32 spaced proximal from the stability element 36, and an explant component 39 at the second portion 34 of the line 32. The decoupling element 38 can be located at either the first portion 33 or the second portion 34 of the line 32. The decoupling element 38, for example, can be located at the first portion 33 of the line 32 adjacent or otherwise close to the stability element 36 so that the second portion 34 of the line 32 has a sufficient length to be adapted for use with shallow or deep implantations. Such a configuration enables a single type of implantable device 20 to be used with different people of different sizes and/or different target depths within the patient. In accordance with alternative embodiments, a decoupling element is not included.

The stability element 36 can be configured to hold the marker 40 at a known position with respect to a target in the tissue such that the marker 40 generally moves corresponding to movement of the target. As such, when respiration or other movement of the patient causes the target to move, the stability element 36 holds the marker 40 in the same position relative to the target so that the marker 40 follows the movement of the target. In accordance with one embodiment, decoupling element 38 is configured to inhibit deformation of the tissue in which the marker 40 is implanted from moving the marker 40 relative to the target. For example, when the skin or other tissue adjacent to the implantable device 20 moves but the target does not move in a corresponding manner, the decoupling element 38 disassociates movement of the line 32 proximal of the decoupling element 38 from the marker 40 to prevent or at least inhibit the marker 40 from moving relative to the target. The explant component 39 is configured to be at least near the dermis 8. For example, the explant component 39 can be fixed to the patient using tape, sutures, glue, buttons, or other suitable means such that the explant component 39 is subdermal (shown in broken lines by reference number 39) or supradermal (shown in solid lines by reference number 39). The explant component 39 is configured to enable quick removal of the marker 40 from the patient 6. As explained in more detail below, the stability element 36, decoupling element 38 and explant component 39, can all be different portions of the line 32.

The marker 40 can have a shell 41 with a nose 42 at a proximal end configured to be attached to the line 32. The shell 41 can further include a recess 43 or other means for receiving a rod used in implanting the implantable device 20. The marker 40 can further include circuitry 44 configured to wirelessly transmit a location signal in response to a wirelessly transmitted source or excitation signal. The circuitry 44, for example, can include a core, a coil, and a capacitor to produce and transmit an alternating magnetic location signal in response to an alternating magnetic source signal. Suitable circuitry 44 for the marker 40 is described in more detail in the U.S. patent applications incorporated by reference above.

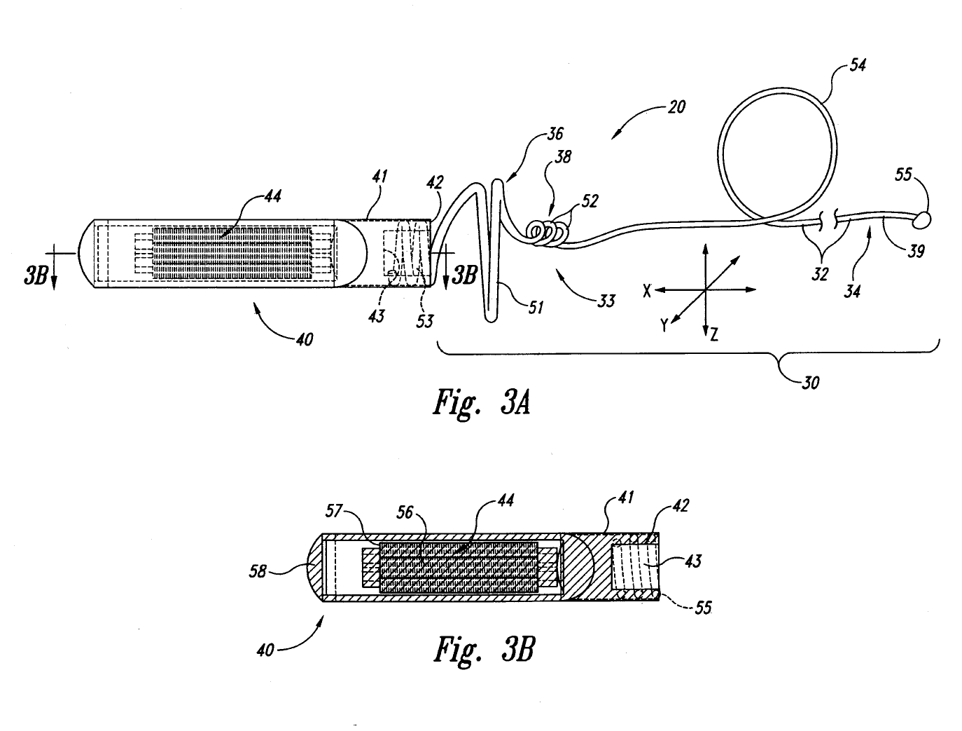

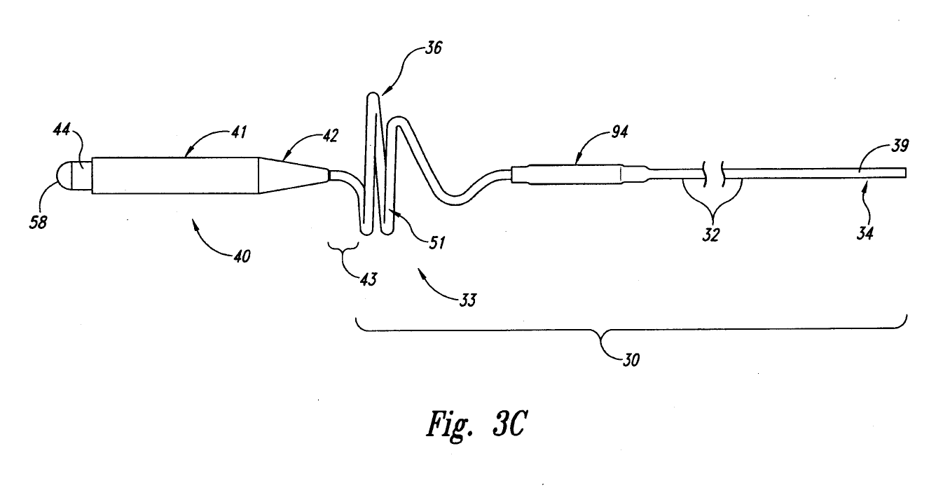

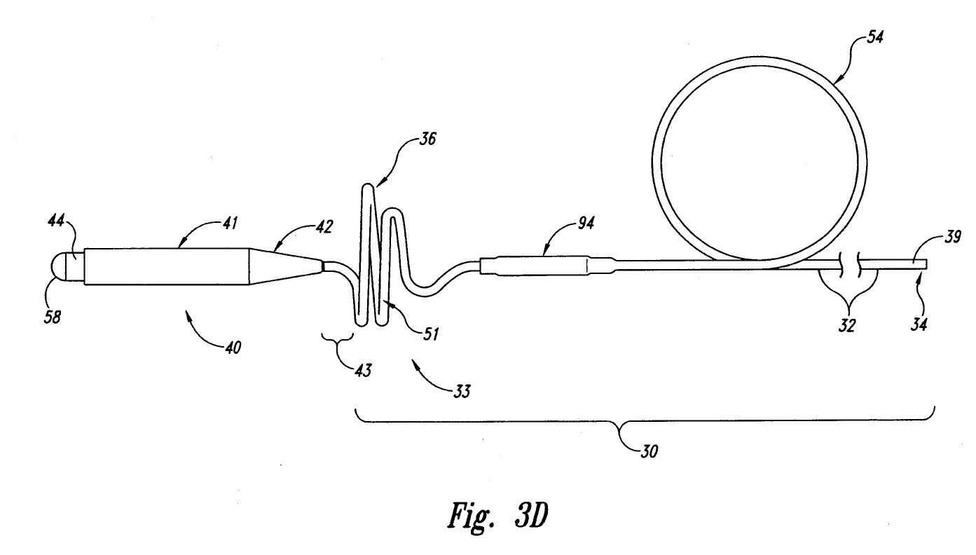

FIG. 3A is a side view of a specific embodiment of the implantable device 20. In this embodiment, the stability element 36 of the fastening unit 30 comprises a first coil 51 and the decoupling element 38 comprises a second coil 52. The first coil 51 can have one or more windings (e.g., loops) with a first loop diameter and/or pitch configured to encompass a relatively large area so that the first coil 51 holds the marker 40 at a constant location distally of the first coil 51. The first coil 51 can be made from a first filament section comprising a polymer, metal, or combination thereof, filaments exhibiting shape memory, superlastic, or their combinations, with a diameter of 0.025″ to 0.250″. In the example shown in FIG. 3A, the first coil 51 has one loop because the first coil 51 is elongated to fit into an introducer (e.g., trocar) and will recoil upon deployment causing the marker 40 to move proximally a short distance. As such, to provide good placement accuracy, several embodiments of the first coil 51 have a limited number of windings to limit the recoil displacement upon deployment. The second coil 52 can have one or more windings with a second loop diameter and/or pitch configured to easily expand/compress longitudinally with respect to the line 32 (e.g., along an X-direction) and flex transversely with respect to the line 32 (e.g., along Y- and Z-directions). The second coil 52 can comprise a second filament section made from a polymer, metal or combination thereof, filaments exhibiting including shape memory, superlastic or their combinations, with a diameter of 0.025″ to 0.250″ with a diameter of 0.015″ to 0.200″. Alternatively, the diameter of the wire may be 10-100% of the diameter of the marker 40, or the wire may be approximately 30% of the diameter of the marker 40. Alternatively, the coil may have a non-cylindrical geometry (e.g., conical, hourglass, or barrel) so as to modify the mechanical properties of the spring. The first and second coils 51-52 can be made from the same material such that the first and second filament sections are integral portions of the same line, or the first and second coils 51-52 can be made from different materials such that the first and second filament sections are separate sections connected together at an interface. In one embodiment, the first coil 51 can have a first flexibility and the second coil 52 can have a second flexibility such that the stability element 36 is less flexible than the decoupling element 38 (e.g., the stability element 36 is relatively rigid but the decoupling element 38 is relatively flexible). In operation, the first and second coils 51-52 function together such that the first coil 51 anchors the marker 40 in the tissue such that the marker 40 moves with the tissue distally of the first coil 51 while the second coil 52 decouples tissue movement proximally of the first coil 51 so that collateral tissue movement does not cause relative displacement between the marker 40 and the target. In an alternative embodiment, a decoupling element is not provided; rather, tissue movement proximal of the first coil 51 is decoupled through a flexible line 32, wherein the flexibility of the line 32 reduces or eliminates translation of motion to the first coil.

The fastening unit 30 can further include a connector 53 configured to engage the recess 43 of the marker 40 and an equilibrium element 54 proximal of the decoupling element 38. In the example shown in FIG. 3A, the connector 53 is a coil at the terminus of the first portion 33 of the line 32 and the equilibrium element 54 is a loop or other configuration of extra line. The equilibrium element 54 reduces the displacement of the marker 40 at deployment caused by the recoil, if any, of the first and second coils 51-52 because the equilibrium element 54 provides additional line proximally of the decoupling element 38 that can be pulled distally as the stability element 36 and the decoupling element 38 recoil. As such, the recoil of the stability and decoupling elements upon deployment does not pull the marker 40 proximally. The equilibrium element 54 can further reduce movement of the proximal second portion 34 of the line 32 at the dermis of the patient to reduce the risk of infection. The proximal second portion 34 of the line 32 can also have a knob 55 or a loop 54 to make it easier to grip and pull the line 32 for extracting the implantable device 20 from the patient. The loop 54 also provides simple coiling of the line for securement at the dermis.

FIG. 3B is a side view of the embodiment of the marker 40 in FIG. 3A. In the example shown in FIG. 3B, the circuitry 44 of the marker 40 has a core 56, a plurality of windings 57 around the core 56, and a casing 58 encapsulating the core 56 and the windings 57. In the example shown in FIG. 3B, the shell 41 has a cavity and the casing 58 is received in the cavity of the shell 41. The shell 41 and casing 58, therefore, can be separate elements that are adhered together. In other embodiments, the shell 41 can be configured to encapsulate the circuitry 44 such that a separate casing is eliminated.

FIG. 3C is a side view of a specific embodiment of the implantable device 20. In the example shown, a flexible line 32 connects a nose 42 at a proximal end of the implant 40 shown in the shape of a cone. The flexible line 32 is constructed from a combination of different materials or constructions or combinations thereof, for example, the flexible line can be a thin mono-filament, multifilament, cable, and/or combination thereof, made from a dielectric material (e.g., a polymer), a metal wire, and/or a ribbon with a dielectric coating. The flexible line 32 is connected to the stability element 36 by means of a joint or coupling. According to aspects of this embodiment, the proximal nose cone 42 is shown in a cone shape, including a 5-15 degree angle. A cone shape on the proximal end of the marker may dilate tissue during removal of the marker and allow for a reduced removal force. As will be appreciated by those skilled in the arts, the angle of the cone may be greater than 15 degrees or less than 5 degrees. As will be further appreciated, the shape of the marker 40 and/or of the proximal nose cone 42 may be round, square, angular, undulating, asymmetrical and/or any other geometrical shape.

FIG. 3D is a cross-sectional view of an alternative embodiment of the implantable device 20 shown with a loop 54 or other configuration of extra line to allow coiling and securing of the line at the dermis. In operation, the loop 54 provides the user external cable management control. According to aspects of this embodiment, no decoupling element is required.

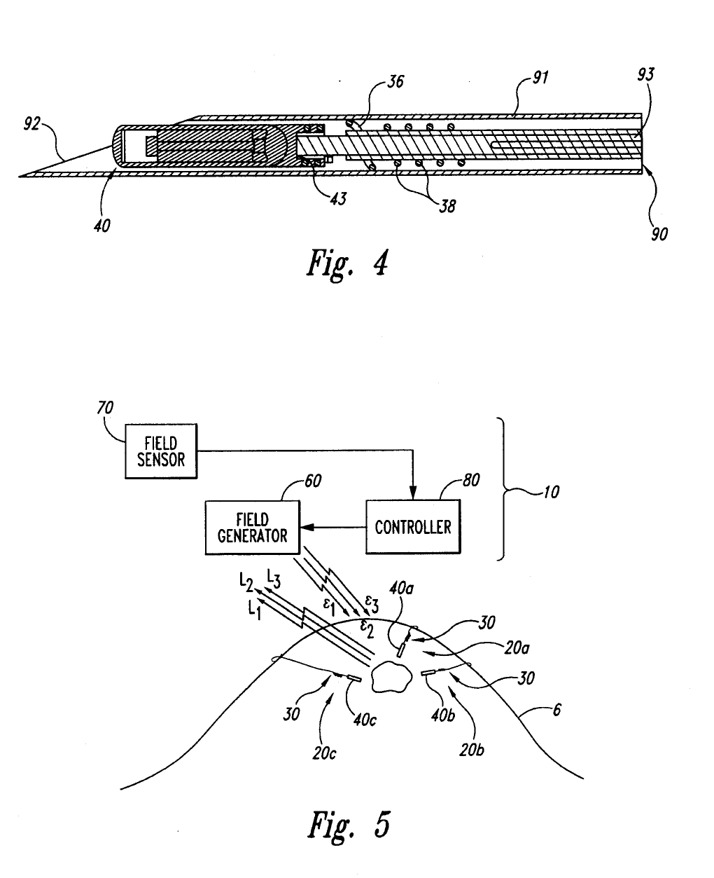

FIG. 4 is a cross-sectional view of an embodiment of the implantable device 20 loaded in an introducer 90 that is configured to implant the implantable device 20 in a patient. In this embodiment, the introducer 90 has a tube 91 (e.g., a needle) with a cutting tip 92 (e.g., a trocar) and a rod 93 (e.g., a stylet) in the tube. The implantable device 20 is loaded in the tube 91, for example, by elongating the first coil 51, with or without elongation of the second coil 52 around the rod 93. The distal terminus of the rod 93 is positioned in the recess 43 of the marker 40. In operation, the tube 91 and loaded implantable device 20 are inserted into the patient using a guidance system, such as ultrasound, until the marker 40 is positioned at a desired location in the tissue relative to the target. The implantable device 20 is then deployed by withdrawing the tube 91 proximally relative to the rod 93 and/or pushing the rod 93 distally relative to the tube 91. The first coil 51 recoils to its deployed configuration shown in FIG. 3B as the tube 91 is withdrawn to operate as explained above. The second coil 52 may or may not recoil depending on the method used to introduce the coil into the tube. The explant component is then attached at or near the dermis of the patient, and the precise location of the marker 40 relative to the target is measured using a CT scan or other imaging modality. Alternatively, the rod 93 has a hollow that allows the filament and elongated coils to pass through a portion or all of its axis.

FIG. 5 is a schematic view illustrating the operation of an embodiment of the localization system 10 and markers 40a-c for treating a target in the breast of the patient. The markers 40a-c are used to determine the location, orientation, shape, size, and/or other parameter of a resection cavity or other target before, during, and after radiation sessions. More specifically, the localization system 10 determines the locations of the markers 40a-c and provides objective target position data to a memory, user interface, linear accelerator, and/or other devices in real time during setup, treatment, deployment, simulation, surgery, and/or other medical procedures. In one embodiment of the localization system, real time means that indicia of objective coordinates are provided to a user interface at (a) a sufficiently high refresh rate (i.e., frequency) such that pauses in the data are not humanly discernable and (b) a sufficiently low latency to be at least substantially contemporaneous with the measurement of the original signal. In other embodiments, real time is defined by higher frequency ranges and lower latency ranges for providing the objective data, or in still other embodiments, real time is defined as providing objective data responsive to the location of the markers (e.g., at a periodicity or frequency that adequately tracks the location of the target in real time and/or at a latency that is at least substantially contemporaneous with obtaining position data of the markers).

The excitation source 60 (e.g., pulsed magnetic field generator), sensor assembly 70, and controller 80 operate together to localize the markers 40. The excitation source 60 generates an excitation energy to energize at least one of the markers 40a-c in the patient 6. The embodiment of the excitation source 60 shown in FIG. 5 produces a pulsed magnetic field at different frequencies. For example, the excitation source 60 can frequency multiplex the magnetic field at a first frequency E1 to energize the first marker 40a, a second frequency E2 to energize the second marker 40b, and a third frequency E3 to energize the third marker 40c. In response to the excitation energy, the markers 40a-c generate location signals L1-3 at unique response frequencies. More specifically, the first marker 40a generates a first location signal L1 at a first frequency in response to the excitation energy at the first frequency E1, the second marker 40b generates a second location signal L2 at a second frequency in response to the excitation energy at the second frequency E2, and the third marker 40c generates a third location signal L3 at a third frequency in response to the excitation energy at the third frequency E3. In an alternative embodiment with two markers, the excitation source generates the magnetic field at frequencies E1 and E2, and the markers 40a-b generate location signals L1 and L2, respectively.

The sensor assembly 70 can include a plurality of coils to sense the location signals L1-3 from the markers 40a-c. The sensor assembly 70 can be a flat panel having a plurality of coils that are at least substantially coplanar relative to each other. In other embodiments, the sensor assembly 70 may be a non-planar array of coils.

The controller 80 includes hardware, software, or other computer-operable media containing instructions that operate the excitation source 60 to multiplex the excitation energy at the different frequencies E1-3. For example, the controller 80 causes the excitation source 60 to generate the excitation energy at the first frequency E1 for a first excitation period, and then the controller 80 causes the excitation source 60 to terminate the excitation energy at the first frequency E1 for a first sensing phase during which the sensor assembly 70 senses the first location signal L1 from the first marker 40a without the presence of the excitation energy at the first frequency E1. The controller 80 then causes the excitation source 60 to (a) generate the second excitation energy at the second frequency E2 for a second excitation period; and (b) terminate the excitation energy at the second frequency E2 for a second sensing phase during which the sensor assembly 70 senses the second location signal L2 from the second marker 40b without the presence of the second excitation energy at the second frequency E2. The controller 80 then repeats this operation with the third excitation energy at the third frequency E3 such that the third marker 40c transmits the third location signal L3 to the sensor assembly 70 during a third sensing phase. As such, the excitation source 60 wirelessly transmits the excitation energy in the form of pulsed magnetic fields at the resonant frequencies of the markers 40a-c during excitation periods, and the markers 40a-c wirelessly transmit the location signals L1-3 to the sensor assembly 70 during sensing phases. It will be appreciated that the excitation and sensing phases can be repeated to permit averaging of the sensed signals to reduce noise.

The computer-operable media in the controller 80, or in a separate signal processor, also includes instructions to determine the absolute positions of each of the markers 40a-c in a three-dimensional reference frame. Based on signals provided by the sensor assembly 70 that correspond to the magnitude of each of the location signals L1-3, the controller 80 and/or a separate signal processor calculates the absolute coordinates of each of the markers 40a-c in the three-dimensional reference frame.

The embodiments of systems and implantable devices for guided radiation therapy described above can be used in methods for treating a patient after a procedure that leaves a resection cavity within the patient. An embodiment of such a method comprises implanting an active marker in tissue of the patient at the resection cavity, wherein the active marker is configured to transmit a non-ionizing wireless signal in response to a non-ionizing wirelessly transmitted source energy, and wherein the marker is coupled to a fastening unit having a distal first portion and a proximal second portion. The method can further include securing the second portion of the fastening unit at least proximate to the dermis of the patient, and localizing the active marker by wirelessly transmitting a non-ionizing source energy to the active marker, transmitting a non-ionizing location signal from the active marker in response to the source energy, and calculating a position of the active marker in an external coordinate system based on the location signal.

### 2. Real Time Tracking

The localization system 10 and markers 40 enable real time tracking of the target and/or status of the resection cavity or other target relative to an external reference frame outside the patient during treatment planning, setup, irradiation sessions, and other times of the radiation therapy process. In many embodiments, real time tracking means collecting position data of the markers, determining the locations of the markers in an external reference frame (i.e., a reference frame outside the patient), and providing an objective output in the external reference frame responsive to the location of the markers. The objective output is provided at a frequency/periodicity that adequately tracks the target in real time and/or a latency that is at least substantially contemporaneous with collecting the position data (e.g., within a generally concurrent period of time).

For example, several embodiments of real time tracking are defined as determining the locations of the markers and calculating the locations relative to an external reference frame at (a) a sufficiently high frequency/periodicity so that pauses in representations of the target location at a user interface do not interrupt the procedure or are readily discernable by a human, and (b) a sufficiently low latency to be at least substantially contemporaneous with the measurement of the location signals from the markers. Alternatively, real time means that the localization system 10 calculates the absolute position of each individual marker 40 and/or the location of the target at a periodicity of approximately 1 ms to 5 seconds, or in many applications at a periodicity of approximately 10-100 ms, or in some specific applications at a periodicity of approximately 20-50 ms. In applications for user interfaces, for example, the periodicity can be 12.5 ms (i.e., a frequency of 80 Hz), 16.667 ms (60 Hz), 20 ms (50 Hz), and/or 50 ms (20 Hz). Additionally, real time tracking can further mean that the localization system 10 provides the absolute locations of the markers 40 and/or the target to a memory device, user interface, linear accelerator, or other device within a latency of 10 ms to 5 seconds from the time the localization signals were transmitted from the markers 40. In more specific applications, the localization system 10 generally provides the locations of the markers 40, target, or an instrument within a latency of about 20-50 ms. The localization system 10 accordingly provides real time tracking to monitor the position of the markers 40 and/or the target with respect to an external reference frame in a manner that is expected to enhance the efficacy of radiation therapy.

Alternatively, real time tracking can further mean that the localization system 10 provides the absolute locations of the markers 40 and/or the target to a memory device, user interface, or other device within a latency of 10 ms to 5 seconds from the time the localization signals were transmitted from the markers 40. In more specific applications, the location system generally provides the locations of the markers 40 and/or target within a latency of about 20-50 ms. The localization system 10 accordingly provides real time tracking to monitor the position of the markers 40 and/or the target with respect to an external reference frame in a manner that is expected to enhance the efficacy of radiation therapy because higher radiation doses can be applied to the target and collateral effects to healthy tissue can be mitigated.

Alternatively, real-time tracking can further be defined by the tracking error. Measurements of the position of a moving target are subject to motion-induced error, generally referred to as a tracking error. According to specific embodiments, the localization system 10 and at least one marker 40 enable real time tracking of the target or other instrument relative to an external reference frame with a tracking error that is within clinically meaningful limits.

Tracking errors are due to two limitations exhibited by any practical measurement system, specifically (a) latency between the time the target position is sensed and the time the position measurement is made available, and (b) sampling delay due to the periodicity of measurements. For example, if a target is moving at 5 cm/s and a measurement system has a latency of 200 ms, then position measurements will be in error by 1 cm. The error in this example is due to latency alone, independent of any other measurement errors, and is simply due to the fact that the target or instrument has moved between the time its position is sensed and the time the position measurement is made available for use. If the measurement system further has a sampling periodicity of 200 ms (i.e., a sampling frequency of 5 Hz), then the peak tracking error increases to 2 cm, with an average tracking error of 1.5 cm.

For a real time tracking system to be useful in medical applications, it is desirable to keep the tracking error within clinically meaningful limits. For example, in a system for tracking motion of a tumor or an instrument for radiation therapy, it may be desirable to keep the tracking error within 5 mm. Acceptable tracking errors may be smaller when tracking other organs for radiation therapy. In accordance with aspects of the present invention, real time tracking refers to measurement of target position and/or rotation with tracking errors that are within clinically meaningful limits.

### 3. Embodiments of Explanting the Implantable Devices

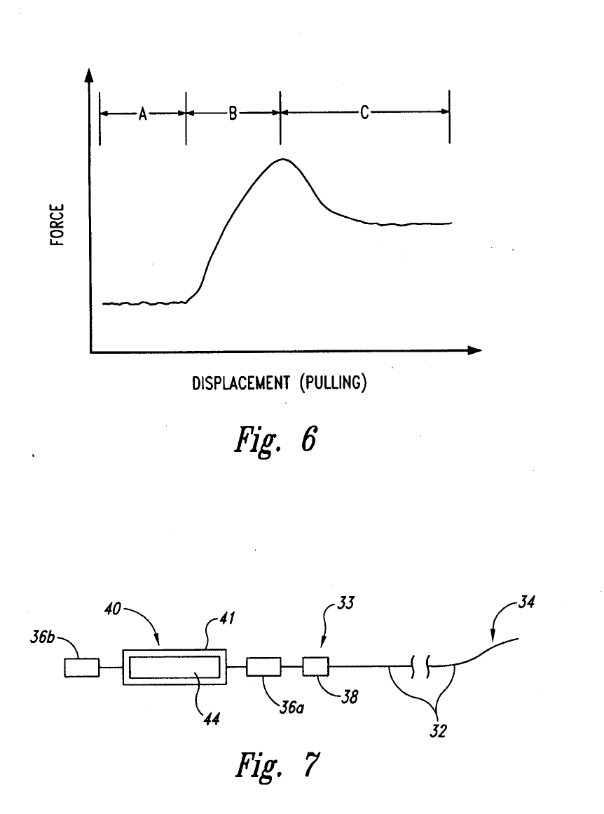

FIG. 6 is a graph illustrating an example of a force curve for explanting the implantable device. To explant the implantable device, a medical practitioner grips the proximal portion of the line and pulls the line proximally. As shown by section A in FIG. 6, the initial force can be relatively low and constant while the decoupling element elongates. Section B in FIG. 6 shows a increase in force relative to the displacement that can correspond to the force loading on the stability element before the stability is dislodged from the local tissue. The stability element is dislodged from the tissue at the demarcation between section B and section C, resulting in a rapid decrease in force relative to the displacement. The force curve shown in FIG. 6 is merely one example of the operation of an embodiment of the implantable device.

The combination of the optional decoupling element and the stability element also can provide a reliable structure for removing the implantable device from the patient. In the embodiment shown in FIG. 6, the rapid increase in force followed by the rapid decrease in force associated with dislodging the stability element provides tactile feedback that enhances the reliability and control of the implantable device. Additionally, the implantable device can be configured to provide comfort to the patient during the explant procedure. For example, smaller filament sizes for the line and smaller diameter coils are generally more comfortable for the patient as the implantable device is pulled out through the tissue. These parameters, however, are balanced against the desire to have larger filament sizes and larger outer diameter coils for the stability element to increase the holding properties of the stability element.

### 4. Additional Embodiments of Apparatus for Facilitating Radiation Treatment

FIG. 7 is a schematic side view of another embodiment of the implantable device 20 for guided radiation therapy. In this embodiment, the implantable device 20 has a first stability element 36a at the proximal end of the marker 40 and a second stability element 36b at the distal end of the marker 40. The first and second stability elements 36a-b can be the same as each other, but in other embodiments the first and second stability elements 36a-b can be different from each other. The first and second stability elements 36a-b, for example, can be single-loop or multiple-loop coils made from a filament having a relatively large coil diameter.

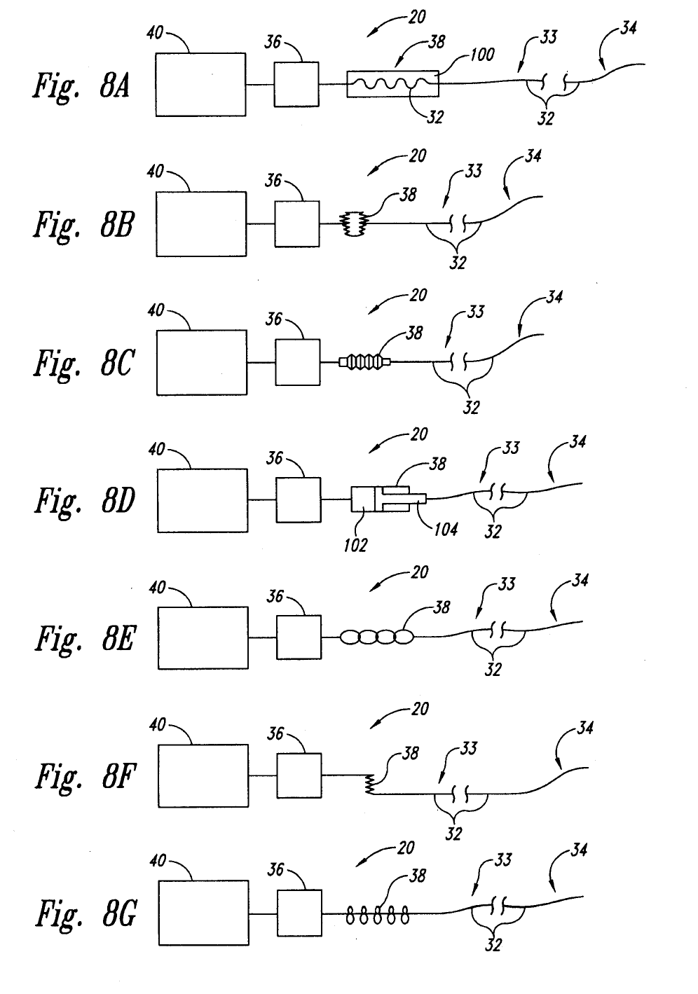

FIGS. 8A-8F are side views of additional embodiments of decoupling elements 38 used in implantable devices for guided radiation therapy. The embodiment of the decoupling element 38 shown in FIG. 8A comprises a flexible casing 100 and excess portion of the line 32 arranged in a serpentine pattern within the flexible casing 100. The flexible casing 100 can be a thin silicone or polymer that can deform longitudinally and/or transversely with respect to the line 32. The embodiment of the decoupling 38 shown in FIG. 8B comprises a loop of a flexible element having a zigzag configuration to enhance the flexibility. The example of the decoupling element 38 shown in FIG. 8C comprises a flexible accordion-like member, and the decoupling element 38 of FIG. 8D comprises a cylinder 102 and a piston 104. The decoupling element 38 of FIG. 8D allows longitudinal displacement along the line 32, but is not generally suitable for lateral displacement with respect to the line 32. FIG. 8E illustrates an embodiment of the decoupling element 38 comprising a fine chain, and FIG. 8F shows an embodiment of the decoupling element 38 having a lateral zigzag filament section. FIG. 8G shows an embodiment of the decoupling element 38 having a crochet or knit filament section.

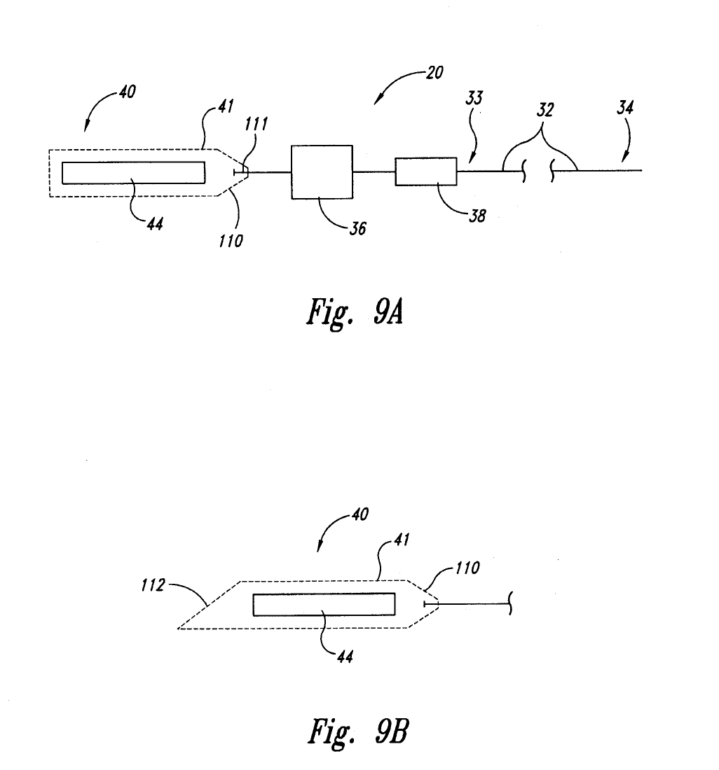

FIGS. 9A and 9B are side views of additional embodiments of markers 40 for implantable devices used in guided radiation therapy. FIG. 9A illustrates an embodiment of the marker 40 in which the shell 41 has a proximal conical nose 110 configured to be pulled proximally through the tissue of the patient and the line 32 has a connector molded in the nose 110. FIG. 9B shows an embodiment of the marker 40 in which the shell 41 has a distal tip 112 configured to match the cutting edge of the tube in which the marker is loaded for implantation. The tip 112 can prevent tissue from entering the tube as the tube and marker are pushed into the tissue.

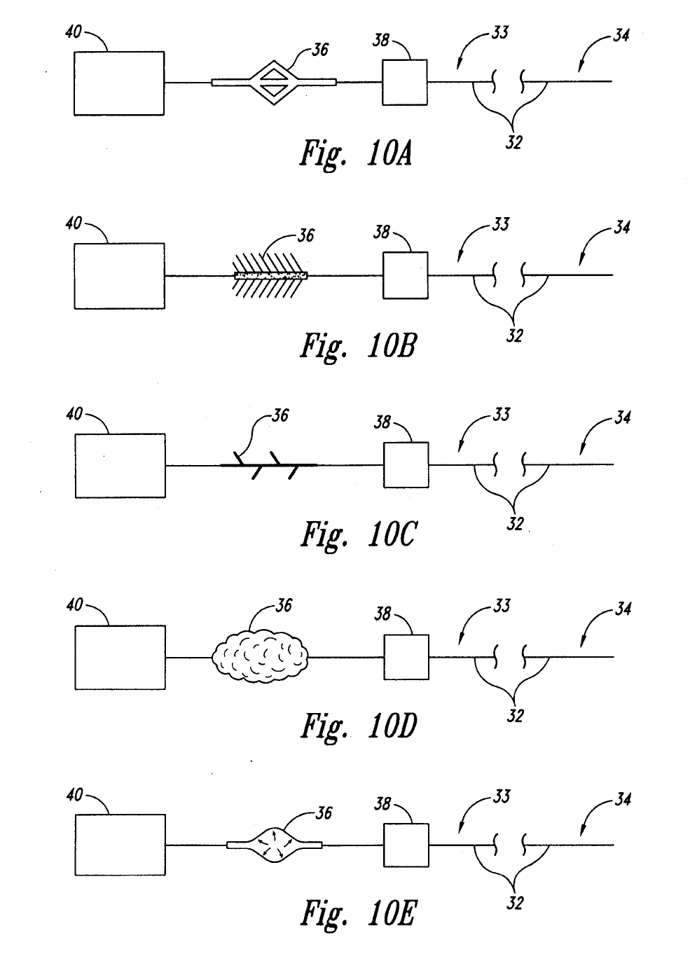

FIGS. 10A and 10E are side views of additional embodiments of stability elements 36 used in implantable devices for guided radiation therapy. FIG. 10A illustrates an embodiment of the stability element 36 comprising an expandable scaffold structure. FIG. 10B shows an embodiment of the stability element 36 having a multitude of short filament structures. FIG. 10C shows and embodiment of the stability element 36 with barb like filaments. FIG. 10D shows an embodiment of the stability element 36 comprising an absorbable structure. FIG. 10 E shows an embodiment of the stability element 36 having an inflated structure.

From the foregoing, it will be appreciated that specific embodiments of the invention have been described herein for purposes of illustration, but well-known structures and functions have not been shown or described in detail to avoid unnecessarily obscuring the description of the embodiments. Where the context permits, singular or plural terms may also include the plural or singular term, respectively. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of features are not precluded. Accordingly, the invention is not limited except as by the appended claims and any claims filed during prosecution and/or in an application claiming priority to the foregoing disclosure.

I/We claim:

1. An implantable device for guided radiation therapy, comprising:
an active marker having a circuit configured to be implanted in a patient and to transmit a wirelessly transmitted location signal from within the patient; and
a fastening unit having a first portion coupled to the active marker and a second portion configured to be at least proximate to the dermis of the patient.

2. The device of claim 1 further comprising a plurality of active markers.

3. The device of claim 1 wherein the active marker comprises a wired marker having a casing and a core within the casing, and wherein the circuit comprises a coil within the casing around the core.

4. The device of claim 1 wherein the active marker comprises a leadless marker having a casing and a core within the casing, and wherein the circuit comprises a coil within the casing around the core.

5. The device of claim 1 wherein the fastening unit comprises a stability element configured to fix the marker to the tissue and a decoupling element configured to inhibit deformation of tissue from moving the marker relative to the target.

6. The device of claim 1 wherein the fastening unit comprises a stability element configured to fix the marker to the tissue without a decoupling element configured to inhibit deformation of tissue from moving the marker relative to the target.

7. The device of claim 5 wherein the stability element comprises a first coil having a first loop diameter and the decoupling element comprises a second coil having a second loop diameter less than the first loop diameter.

8. The device of claim 7 wherein the first coil has a first flexibility and the second coil has a second flexibility greater than the first flexibility.

9. The device of claim 7 wherein the first coil is made from a first material and the second coil is made from a second material and construction different than the first material.

10. The device of claim 7 wherein the first coil is made from a first material and the second coil is made from the same material wherein the material of the second coil is processed differently than the material of the first coil.

11. The device of claim 7 wherein the first coil has a first filament diameter and the second coil has a second filament diameter less than the first filament diameter.

12. The device of claim 7 wherein the first coil has not more than two loops and the second coil has at least two loops.

13. The device of claim 12 wherein the first coil has a single-loop.

14. The device of claim 7 wherein the first coil has not more than 10 loops.

15. The device of claim 5 wherein the decoupling element comprises a coil.

16. The device of claim 5 wherein the decoupling element comprises a flexible casing and a serpentine section of filament.

17. The device of claim 5 wherein the decoupling element comprises a loop of filament having a zigzag configuration.

18. The device of claim 5 wherein the decoupling element comprises an accordion-like member.

19. The device of claim 5 wherein the decoupling element comprises a piston and a cylinder.

20. The device of claim 5 wherein the decoupling element comprises a chain.

21. The device of claim 5 wherein the decoupling element comprises a lateral zigzag member.

22. The device of claim 5 wherein the decoupling element comprises a crochet loop member.

23. The device of claim 1 wherein the marker comprises a shell having a conical proximal nose.

24. The device of claim 1 wherein the marker comprises a shell having a tip with a conical surface.

25. The device of claim 1 wherein the marker comprises a shell having a tip with an inclined surface.

26. An implantable device for guided radiation therapy, comprising:
an active marker configured to be implanted in tissue of the patient and to transmit a non-ionizing wireless signal;
a stability element coupled to the marker and configured to hold the marker within a desired distance of a target in the tissue; and
an explant line having a first portion coupled to at least one of the marker and the stability element and a second portion configured to be at least proximate to the dermis of the patient.

27. The device of claim 26, further comprising a decoupling element coupled to the explant line and spaced proximally apart from the stability element, wherein the decoupling element is configured to inhibit deformation of tissue from moving the active marker relative to the target.

28. The device of claim 27 wherein the stability element comprises a first coil having a first loop diameter and the decoupling element comprises a second coil having a second loop diameter less than the first loop diameter.

29. The device of claim 28 wherein the first coil has a first flexibility and the second coil has a second flexibility greater than the first flexibility.

30. The device of claim 28 wherein the first coil is made from a first material and the second coil is made from a second material and construction different than the first material.

31. The device of claim 28 wherein the first coil has a first filament diameter and the second coil has a second filament diameter less than the first filament diameter.

32. An implantable device for guided radiation therapy, comprising:
an active marker configured to be positioned within the patient and to transmit a non-ionizing wireless signal; and
a fastening unit coupled to the active marker and configured to (a) hold the marker within a desired distance of a target in the tissue and (b) inhibit deformation of tissue from moving the active marker relative to the target.

33. The device of claim 32 wherein the stability element comprises a first coil having a first loop diameter and the decoupling element comprises a second coil having a second loop diameter less than the first loop diameter.

34. The device of claim 33 wherein the first coil has a first flexibility and the second coil has a second flexibility greater than the first flexibility.

35. The device of claim 33 wherein the first coil is made from a first material and the second coil is made from a second material and construction different than the first material.

36. The device of claim 33 wherein the first coil has a first filament diameter and the second coil has a second filament diameter less than the first filament diameter.

37. A method for treating a patient, the method comprising:
implanting an active marker in tissue of the patient at or near a resection cavity, wherein the active marker is configured to transmit a non-ionizing wireless signal in response to a non-ionizing wirelessly transmitted source energy, and wherein the marker is coupled to a fastening unit having a distal first portion and a proximal second portion;
securing the second portion of the fastening unit at least proximate to the dermis of the patient; and
localizing the active marker by wirelessly transmitting a non-ionizing location signal from the active marker in response to the source energy, and calculating a position of the active marker in an external coordinate system based on the location signal.

38. The method of claim 37, further comprising directing an external radiation beam toward the resection cavity based on the calculated position of the active marker.

39. The method of claim 38, further comprising terminating the radiation beam and pulling the fastening unit after terminating the radiation beam until the marker is removed from the patient.
